(19) United States
(12) Patent Application Publication
(10) Pub. No.: US 2010/0261726 A1
(43) Pub. Date: Oct. 14, 2010

(54) TREATMENT OF AML

(76) Inventors: Eveline S.J.M. De Bont, Groningen (NL); Willem A. Kamps, Winsum (NL)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/423,847

(22) Filed: Apr. 15, 2009

Related U.S. Application Data
(63) Continuation of application No. 10/530,452, filed on Sep. 9, 2005, now abandoned.

(30) Foreign Application Priority Data
Oct. 8, 2002 (GB) 0223341.9

(51) Int. Cl. A61K 31/501; A61K 31/7068; A61P 35/02

(52) U.S. Cl. 514/252.01; 514/49

(57) ABSTRACT

The present invention relates to a method of treating a warm-blooded animal having acute myeloid leukemia (AML) which is resistant to conventional chemotherapy, comprising administering to said animal a therapeutically effective amount of a compound of formula I

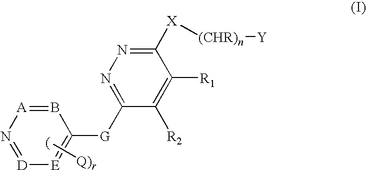

wherein the radicals and symbols have the meanings as defined in the specification, together or in combination with a conventional compound or compound mixture useful in AML treatment, in particular a topoisomerase II inhibitor, an antimetabolite, or an antitumor antibiotic, for simultaneous, separate or sequential use, and to a pharmaceutical composition and a commercial package comprising said combination.

The present invention relates to a method of treating a warm-blooded animal, especially a human, having leukemia, especially acute myeloid leukemia (AML), in particular acute myeloid leukemia which is resistant to conventional chemotherapy, comprising administering to said animal a therapeutically effective amount of a compound of formula I as defined herein; together or in combination with a conventional compound or compound mixture useful in AML treatment, in particular a topoisomerase II inhibitor, an antimetabolite, or an antitumor antibiotic, and optionally at least one pharmaceutically acceptable carrier, for simultaneous, separate or sequential use; and to a pharmaceutical composition and a commercial package comprising said combination.

### SHORT DESCRIPTION OF THE FIGURES

FIG. 1

Results of RT-PCR (Reverse transcription polymerase chain reaction) of Huvec, U937, TF-1 and HL-60 cell lines and patient leukemic cell samples 1, 2, 3, 5, and 6, demonstrating presence or absence of VEGF, VEGFR-1, VEGFR-2 and β₂-microglobulin.

FIG. 2

Survival assay for TF-1 cells and HL-60 cells on addition of exogenous VEGF counted by trypan blue exclusion after 24 hours.

FIG. 3

Survival assay for TF-1 cells on addition of exogenous VEGF with or without PTK787 counted by trypan blue exclusion after 24 hours.

FIG. 4

Leukemic cell survival of three AML cell lines (HL-60, TF-1 and K562) on addition of PTK787 in different concentrations measured by total cell kill assay.

FIG. 5

A: Increased patient leukemic cell survival on addition of exogenous VEGF counted by trypan blue exclusion

B: Decreased patient leukemic cell survival on addition of PTK787 counted by trypan blue exclusion

FIG. 6

Patient leukemic cell survival on addition of PTK787 measured with total cell kill assay after 72 hours

FIG. 7

LC 50 values of patient leukemic cells for cytarabine and mitoxantrone on addition of PTK787 measured with total cell kill assay after 72 hours

The term “acute myeloid leukemia” as used herein relates to an uncontrolled, quickly progressing growth of myeloid cells, e.g. granulocytes, as well as erythroid and megakaryotic cells and progenitors. In patients with AML the immature myeloid, erythroid or megakaryotic cells severely outnumber erythrocytes (red blood cells) leading to fatigue and bleeding, and also to increased susceptibility to infection. In children as well as in adults AML has a poor prognosis despite the use of aggressive chemotherapeutic protocols. Overall survival rates are 40-60%. Autologous bone marrow transplant preceded by myeloablative chemotherapy does not change the survival but an allogeneic bone marrow transplant preceded by aggressive chemotherapy might increase the survival rates up to 70%. Unfortunately, the availability of a matched sibling donor is limited. Therefore, new therapeutic strategies in AML treatment are necessary.

AML cells are suspected to express VEGF, and AML derived VEGF production is connected with the clinical outcome of AML (ESJM de Bont et al., Br. J. Haematol. 113: 296, 2001). The present invention is based on the hypothesis that leukemic cells might express functional VEGFRs (vascular endothelial growth factor receptors) in addition to VEGF (vascular endothelial growth factor) expression, and that VEGFR expression not only is engaged in the increase of leukemic cell survival but also is engaged in the sensitivity of leukemic cells for chemotherapeutic agents. In response to leukemic cell derived VEGF, endothelial cells in the bone marrow may release growth factors that support the growth of leukemic cells in a paracrine way. If certain leukemic cells acquire the capacity to express functional VEGFRs, this might generate an autocrine loop.

VEGF, now known as VEGF-A, is one of the best-studied and characterized inducers of angiogenesis. VEGF appears to be a potent stimulator of endothelial cell migration and proliferation. VEGF can bind to fit-1/VEGFR-1 and flk-1/KDRNEGFR-2 but not to flt-4/VEGFR-3, all belonging to the class of tyrosine kinase receptors. Whereas both VEGFR-1 and -2 bind VEGF-A with high affinity, VEGFR-2 is the main receptor to activate a mitogenic response and VEGFR-1 might participate in cell migration.

The compounds of formula I as defined herein and, in particular, PTK787 (also known as ZK222584) are tyrosine kinase inhibitors which were designed to inhibit the vascular endothelial growth factor (VEGF) signal transduction by binding directly to the ATP-binding sites of VEGFRs. The drug is most specific for KDR, but can also inhibit fit-1 and fit-4 and has activity against other tyrosine kinase receptors, including c-kit. PTK787 inhibits the growth of several human carcinomas transplanted orthotopically into mice, including the A431 epidermoid carcinoma, Ls174T colon carcinoma, HT-29 colon carcinoma, and PC-3 prostate carcinoma as described by J. Wood et al., Cancer Res. 60: 2178, 2000. PTK787 does not have a direct effect on any of these tumor cells, but does reduce vessel density in the tumor tissues, suggesting that its primary mode of action in these cells is through inhibition of angiogenesis.

Surprisingly, it was now found that the compounds of formula I as defined herein and, in particular PTK787, efficiently inhibit the proliferation of AML cell lines and patient AML cells that express VEGF and/or VEGFR when combined with conventional compounds useful in the treatment of AML, even when these AML cells are resistant to conventional compounds alone.

Hence, in a first aspect the invention relates to a method of treating acute myeloid leukemia (AML), in particular acute myeloid leukemia which is resistant to conventional chemotherapy, comprising administering to a warm-blooded animal, preferably a human, in need thereof a therapeutically effective amount of a conventional compound useful in AML treatment together or in combination with a compound of formula I

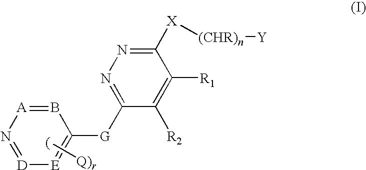

wherein
r is 0 to 2,
n is 0 to 2,
m is 0 to 4,
R₁ and R₂ (i) are lower alkyl or
(ii) together form a bridge in subformula I*

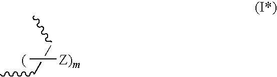

the binding being achieved via the two terminal carbon atoms, or
(iii) together form a bridge in subformula I**

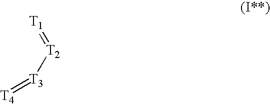

wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
   and wherein the bonds characterized, if present, by a wavy line are either single or double bonds;
   or an N-oxide of the defined compound, wherein 1 or more N atoms carry an oxygen atom, or the salt of such compound having at least one salt-forming group.

The radicals and symbols as used in the definition of a compound of formula I have the meanings as disclosed in WO 98/35958 which publication is hereby incorporated into the present application by reference. In particular those compounds designated as being preferred in WO 98/35958 are also preferred in the present invention.

The term “PTK787” as used herein means a compound of formula I wherein r, n and m are each 0, R₁ and R₂ together form a bridge of subformula I*, A, B, D and E are each CH, G is methylene, X is imino, Y is 4-chlorophenyl, and the bonds characterized by a wavy line are double bonds.

A preferred compound in the treatment of AML together or in combination with a conventional compound useful in the treatment of AML is the compound PTK787 as defined hereinbefore. More preferably, PTK787 is employed in the form of its succinate salt.

Compounds that are also preferred for the treatment of AML according to the present invention are those generically or specifically disclosed, mentioned or generically and specifically claimed in EP 1 259 487, WO 01/55114, EP 1 129 075, WO 00/27820, EP 1 107 964, WO 00/09495, EP 1 165 085, WO 00/59509, WO 02/090343, WO 01/85715, WO 01/85691, WO 02/092603, WO 03/040101 and WO 03/040102, the entire contents of which hereby are incorporated by reference.

In particular compounds which target, decrease or inhibit the activity or production of VEGF are those compounds, proteins, aptamers or monoclonal antibodies generically and specifically disclosed in WO 98/45331, WO 98/11223, WO 00/27819 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999; in WO 00/37502 and WO 94/10202; Angiostatin™, described by M. S. O'Reilly et al, Cell 79, 1994, 315-328; Endostatin™, described by M. S. O'Reilly et al, Cell 88, 1997, 277-285; anthranilic acid amides; ZD4190; ZD6474; SU5416; SU6668; anti-VEGF antibodies or anti-VEGF receptor antibodies, e.g. RhuMab; or those as described in U.S. Pat. No. 6,168,778, U.S. Pat. No. 6,147,204, U.S. Pat. No. 6,051,698, U.S. Pat. No. 6,011,020, U.S. Pat. No. 5,958,691, U.S. Pat. No. 5,817,785, U.S. Pat. No. 5,811,533, U.S. Pat. No. 5,696,249, U.S. Pat. No. 5,683,867, U.S. Pat. No. 5,670,637, and U.S. Pat. No. 5,475,096, e.g. pegaptanib sodium, are also preferred for the treatment of AML according to the present invention.

It will be understood that in the discussion of methods, references to the active ingredients are meant to also to include the pharmaceutically acceptable salts. If these active ingredients have, for example, at least one basic center, they can form acid addition salts. The active ingredients having an acid group (for example COOH) can also form salts with bases. The active ingredient or a pharmaceutically acceptable salt thereof may also be used in form of a hydrate or include other solvents used for crystallization.

The term “treatment” as used herein comprises the treatment of patients having AML or being in a pre-stage or a post-remission stage of said disease. The treatment effects the delay of progression or the partial or complete elimination of the disease in said patients.

Particularly preferred is the treatment according to the invention when applied to juveniles. Hence, a preferred aspect of the invention relates to a method of treating acute myeloid leukemia (AML), in particular acute myeloid leukemia which is resistant to conventional chemotherapy, comprising administering to a juvenile human in need thereof a therapeutically effective amount of a conventional compound useful in AML treatment together or in combination with a compound of formula I as defined hereinbefore, in particular the compound PTK787.

Conventional compounds useful in the treatment of acute myeloid leukemia (AML) comprise, but are not limited to, topoisomerase II inhibitors, such as amsacrine, etoposide or teniposide, anitmetabolites, such as cytarabine, methotexate or mercaptopurine, or antitumor antibiotics, such as mitoxantrone, dactinomycin, daunorubicin, doxorubicin, epirubicin, homoharringtonine or idarubicin. Other conventional compounds useful in the treatment of AML considered in this invention are compounds usually applied in the treatment of ALL (acute lymphoblastic leukemia), such as asparaginase, cyclophosphamide, gemtuzumab (or any other CD 33 monoclonal antibody), ifosfamide, mesna, prednisone, topotecan, and vincristine. Under the term “conventional compound” also mixtures of the mentioned compounds are understood, e.g. combinations of cytarabine with mitoxantrone, amsacrine, daunorubicin, etoposide or idarubicin, or of cytarabine with etoposide and daunorubicin or mitoxantrone. The method of the invention then is characterized by a triple or quadruple therapy comprising administering a compound of formula I together or in combination with two or three conventional compounds useful in the treatment of AML.

Preferred conventional compounds used in the invention are amsacrine, etoposide, cytarabine, daunorubicin and mitoxantrone, and mixtures thereof, in particular amsacrine, cytarabine and mitoxantrone. Particularly preferred are conventional compounds for pediatric use.

The method of administering a compound of formula I together or in combination with a conventional compound or several conventional compounds may further comprise one or more pharmaceutically acceptable carrier, and may involve simultaneous, separate or sequential application of the compounds.

In a further aspect the invention relates to a combined pharmaceutical preparation, especially a “kit of parts” in the sense that the active ingredients as defined above can be dosed independently or by use of different fixed combinations with distinguished amounts of the ingredients, i.e., simultaneously or at different time points. The parts of the kit can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the active ingredients. The ratio of the total amounts of the active ingredient of formula I to the active ingredient(s) of the conventional compound useful in the treatment of AML to be administered in the combined preparation can be varied, e.g., in order to cope with the needs of a patient sub-population to be treated or the needs of the single patient, which different needs can be due to age, sex, body weight, etc. of the patients. Especially preferred is an administration regime taking into account special needs of pediatric use. Preferably, there is at least one beneficial effect, e.g., a mutual enhancing of the effect of the compound of formula I and of the conventional compound, in particular a synergism, e.g. a more than additive effect, additional advantageous effects, less side effects, a combined therapeutical effect in a non-effective dosage of one or each of the active ingredients, and especially a strong synergism of the compound of formula I and the conventional compound useful in the treatment of AML.

The person skilled in the pertinent art is fully enabled to select relevant test models to prove the hereinbefore and hereinafter mentioned beneficial effects on treatment of AML of a compound of formula I together or in combination with a conventional compound useful in the treatment of AML. The activity of the single compounds or of a combination of the invention may, for example, be demonstrated in a suitable clinical study or by means of the Examples described below. Suitable clinical studies are, for example, open label non-randomized, dose escalation studies in patients with advanced AML, preferably in juvenile patients. Such studies prove in particular the synergism observed with the combinations of the invention. The beneficial effects on treatment of AML can be determined directly through the results of such studies or by changes in the study design which are known as such to a person skilled in the art. For example, one combination partner can be administered with a fixed dose and the dose of a second combination partner is escalated until the Maximum Tolerated Dosage (MTD) is reached. Alternatively, a placebo-controlled, double blind study can be conducted in order to prove the benefits of the combination of the invention mentioned herein.

In a further aspect the invention also concerns pharmaceutical compositions. The pharmaceutical compositions for separate administration of the combination partners and for the administration in a fixed combination, i.e. a single galenical composition comprising at least two combination partners, according to the invention can be prepared in a manner known per se and are those suitable for enteral, such as oral or rectal, and parenteral administration to warm-blooded animals, including man, comprising a therapeutically effective amount of at least one pharmacologically active combination partner alone or in combination with one or more pharmaceutically acceptable carries, especially suitable for enteral or parenteral application.

Novel pharmaceutical compositions contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of treatment of AML according to the present invention may comprise (i) administration of a first combination partner in free or pharmaceutically acceptable salt form, (ii) administration of a second combination partner in free or pharmaceutically acceptable salt form, and, optionally (iii) administration of a third, forth, etc. combination partner in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the combination of the invention can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of the compounds of formula I and of the combination partners employed in the combination of the invention may vary depending on the particular compound or pharmaceutical composition employed, the mode of administration, the stage of progression of the AML being treated, the severity of the AML being treated, and the responsiveness of the patient being treated. Thus, the dosage regimen of the combination of the invention is selected in accordance with a variety of factors including the route of administration and the renal and hepatic function of the patient. A physician, clinician or veterinarian of ordinary skill can readily determine and prescribe the effective amount of a compound of formula I or of the other single active ingredients of the combination of the invention required to prevent, counter or arrest the progress of the condition. Optimal precision in achieving concentration of the active ingredients within the range that yields efficacy without toxicity requires a regimen based on the kinetics of the active ingredients' availability to target sites.

If the warm-blooded animal is an adult human, the dosage of a compound of formula I, especially PTK787, is preferably in the range of about 100 to 1500, more preferably about 250 to 1250, and most preferably 500 to 1000 mg/day. In the combination of the invention, the dosage of the compound of formula I may be reduced compared to single application. The dosage of PTK787, for example, in a combination or together with a conventional compound useful in the treatment of AML is preferably in the range of 50 to 1000, more preferably about 100 to 800, and most preferably 200 to 500 mg/day. The preferred dosage of the other combination partner, i.e. the conventional compound for treatment of AML, is also reduced accordingly, preferably to around 50% of the standard dosage.

For use with children, the dosage is reduced accordingly, and adapted to the body weight and/or surface area of the juvenile. For example the dosage of PTK787 in a combination or together with a conventional compound useful in the treatment of AML is preferably in the range of 1 to 15, more preferably about 1.5 to 10, and most preferably 2.5 to 6 mg/day and kg body weight of the patient.

Further, the present invention relates to the use of a pharmaceutical composition as described hereinbefore for the treatment of AML.

Moreover, the present invention provides a commercial package comprising as active ingredients the combination of the invention, together with instructions for simultaneous, separate or sequential use thereof in the treatment of AML.

The present invention also provides the use of a compound of formula I as defined herein and the use of a combination of the invention for the preparation of a medicament for the treatment of AML.

### EXAMPLES

### General Methods and Materials

### Cell Lines and Patient Samples:

The cell lines HL-60, TF-1, K562 and U937 are obtained from American Type Culture Collection (Manassas, Va., USA) and cultured in RPMI-1640 (Roswell Park Memorial Institute) supplemented with penicillin/streptomycin and 10% fetal bovine serum (FBS, Hyclone, Logan, Utah, USA) for U937, K562 and HL-60 cells and supplemented with GM-CSF 1 ng/mL for TF-1 cells.

After informed consent bone marrow and peripheral blood cells were obtained at diagnosis from 6 pediatric AML patients aged 0-18 years. The diagnosis is assessed by cytomorphology using FAB classification and immunophenotyping (Bennett J M et al., Br. J. Haematol. 33: 451-458, 1976). Mononuclear cells (MNC) are separated by Lymphoprep (Nycomed, Oslo, Norway) density gradients and cryopreserved in liquid nitrogen until use. Cryopreserved AML cells are thawed rapidly at 37° C., diluted in a 5× volume of normal calf serum (NCS) as described by Dokter W H et al., Leukemia 9: 425-432, 1995. The remaining pellet is T-cell depleted by sheep red blood cells and separated over lymphoprep density gradient. Remaining blast cell population contained more than 95% AML cells, hereafter referred to as AML cells and cultured in RPMI-1640 supplemented with penicillin/streptomycin and 10% fetal bovine serum.

### Culture of Primary Leukemic Cells and of Leukemic Cell Lines:

Before incubation with recombinant (rec) VEGF₁₆₅ (Sigma, St. Louis, Mich.) and/or PTK787, AML cells and/or cell lines are serum starved for 4 hours in serum free medium (X-vivo 10, Biowhitaker, Brussels, Belgium). For proliferation experiments, cells are cultured in six-well plates (Corning-Costar Corp., Cambridge, Mass., USA), at a cell density of 1×10⁵ cells per well in serum free medium (X-vivo-10) for 24 hours. Cells are treated (rec VEGF₁₆₅ 5-100 ng/mL) or untreated (media alone) and are cultured in the presence or absence of PTK787 (5-100 nM). After 24 hours viable cells (determined by trypan blue exclusion) are counted in triplicate using a hemocytometer. Each experiment is done in triplicate, and experiments with leukemic cell lines are repeated three times.

### Cell Cycle Analysis:

Cells are washed with PBS and resuspended in 75% ethanol in PBS and kept at 4° C. for at least 30 minutes. Prior to analysis, cells are washed with PBS, resuspended and incubated for 30 minutes in 1 mg/mL RNase A in PBS and thereafter incubated in staining solution containing 0.05 mg/mL propidium iodide (Sigma), 1 mM EDTA and 0.1% Triton-X-100. The suspension is then passed through a nylon mesh filter and analyzed on Becton Dickinson FACScan.

### RNA Extraction and RT-PCR:

Total RNA is extracted by the Trizol method following the manufacturer's description (Life Technologies, Gibco BRL, Grand Island, N.Y., USA). cDNAs are prepared by reverse transcription at 37° for at least one hour in a 20 μL reaction mixture containing 2 μg of total RNA, random hexamers (Pharmacia), 5× first strand buffer, RNasin and 1 μL reverse transcriptase (Gibco BRL, Grand Island, N.Y., USA). cDNA is amplified in the presence of primers, 10× buffer, 1.5 mM MgCl₂, dNTPs and Taq (Gibco BRL, Grand Island, N.Y., USA). The mixture is amplified in a Perkin Elmer apparatus with PCR cycle conditions specific for the PCRs tested. PCR product is analyzed by electrophoresis in a 1.5% agarose gel. Gels are stained with ethidium bromide and photographed. Specific primers for β₂-microglobulin are sense (CCA GCA GAG MT GGA AAG TC) and anti-sense (GAT GCT GCT TAC ATG TCT CG), PCR product: 260 bp, 22 cycli, Tann 55° C. For VEGF. sense (GAG TGT GTG CCC ACT GAG GAG TCC MC) and anti-sense (CTC CTGO CCC GGC TCA CCG CCT CGG CTT), PCR product: 177, 312, 384 bp, 30 cycli, Tann 60° C. are used. The primers for VEGF span the splice junctions allowing the amplified product of each splice variant to be separated electrophorically. Specific primers for VEGFR-1: sense (GAG TCC TTT ATC CTG GAT GC) and anti sense (ACA GAG CCC TTC TGG TTG GT), PCR product: 750 bp, 35 cycli, Tann 57° C.

Nested RT-PCR for VEGFR-2: first PCR: sense (CGC TGG GAG AAA GAA CCG) and anti-sense (GCT CAC TGC CAC TCT GAT TAT TG), PCR product: 329 bp, 25 cycli, 1 mM MgCl₂, 5% DMSO, Tann 60° C. Nested PCR: sense (TCC GCG CCT CCT CCT TCT CTA GAC AG) and anti-sense (GGC CAT CGC TGC ACT CAG TGA), PCR product: 270 bp, 25 cycli, 1.25 mM MgCl₂, Tann 53° C., using 4 μL of the first PCR product.

To control for the addition of cDNA in the first PCR reaction, β₂-microglobulin PCR is performed from the same first PCR product as used for the second VEGFR-2 PCR (nested). The first product is split; 4 μL are used for the β₂-microglobulin PCR to control for cDNA addition in the first PCR.

### Protein Extraction and Western Blotting:

Cell lysates are prepared in sample buffer (2% sodium-dodecyl-sulphate, 10% glycerol, 2% β-mercaptho-ethanol and 60 nM Tris-Cl pH 6.8 in demineralized water) on ice. Proteins are resolved on 12.5-15% SDS-polyacrylamide gels (Biorad) and blotted onto PVDF membranes (Millipore, Bedford). Blots are subsequently blocked with 5% non-fat dry milk and 0.05% Tween-20 in Tris-buffered saline (TBST) followed by incubation with primary and secondary antibodies. Polyclonal goat-anti-human VEGFR-2 (Santa Cruz Biotechnology Inc, Santa Cruz, Calif.) antibodies are used, and secondary rabbit-anti-goat HRPO are used (DAKO, Denmark). Proteins are visualized by ECL chemiluminescence detection system and ECL film (Amersham Pharmacia Biotech).

### Cellular Drug Resistance Measurement Using a Total Cell Kill Assay:

In vitro cellular drug resistance of leukemic HL-60 and TF-1 cells (10,000 cells/well) are assessed using a 2-day cell culture assay based on the principle that only viable cells are able to reduce 3-(4,5-dimethyl-2-thiazolyl)-2,5-diphenyl-tetrazolium bromide (MTT) (5 mg/mL in PBS added to each well for 4 hours) to a colored formazan product, measured spectrophotometrically at 520 nm. For the leukemic samples (10,000 cells/well) of the patients a 3-day cell culture is used. Cytarabine (0.001-10 mg/mL) and mitoxantrone (0.0001-1 μg/mL) or doxorubicin (0.0001-1 μg/mL) are tested, each at 9 different concentrations in quadruplicate in 96-well microculture plates. The optical density (OD) is linearly related to the number of viable cells. Control wells contain leukemic cells only with culture media without drugs, and blank wells contain culture media only. Percentage of cell survival is calculated at each drug concentration by the equation (mean OD treated wells/mean OD control wells)×100% after correction for the background found in the blank wells. The results are considered evaluable when the control wells still contain 70% or more leukemic cells (determined by MGG staining) after a 3-day culture period. The mean OD of the control wells after correction for the background at 3 days always exceeds 0.1 arbitrary units for valid results. The LC50 value (drug concentration needed to kill 50% of the leukemic cells) is used to compare the differences between patients and/or various drug combinations. LC50 value equation: ([% leukemic cell survival >50%]−50)/([% leukemic cell survival >50%]−[% leukemic cell survival <50%])×(drug concentration when leukemic cell survival <50%−drug concentration when leukemic cell survival >50%)+(drug concentration when leukemic cell survival >50%).

### Statistical Analysis:

Correlations are calculated using the Spearman rank correlation coefficient (rho). A paired sample non-parametric test (Wilcoxon Signed Ranks test) is used to analyze the paired viable cells (with or without VEGF, as well as with or without PTK787). A significant difference is defined as a P-value <0.05.

### Example 1

### Influence of VEGF and VEGFR on Cell Growth of Leukemic Cell Lines HL-60 and TF-1

The expression of VEGF, VEGFR-1 and VEGFR-2 is examined in TF-1, U937 and HL-60 cells by RT-PCR (FIG. 1) and by a functional assay (FIG. 2). All three cell lines show VEGF expression. HL-60 and U937 cells show positive PCR bands in RT-PCR for VEGFR-1. None of the cell lines tested demonstrates a PCR band for VEGFR-2.

To demonstrate a functional VEGFR, recombinant (rec) VEGF₁₆₅ is added to HL-60 and TF-1 cells in the absence of exogenous growth factors (serum free conditions), and viable cells are counted by trypan blue exclusion after 24 hours stimulation. With rec VEGF₁₆₅ a dose-dependent enhanced leukemic cell survival up to 450% of control cells (100%) is found in TF-1 cells. In HL-60 cells rec VEGF₁₆₅ induces an enhanced cell survival up to 980% of control cells (FIG. 2).

The importance of VEGFNEGFR signaling can be underlined when interruption of VEGFR signaling by PTK787 decreases cell survival. TF-1 cell survival is decreased to 21.9% when 25 nM PTK787 is added (FIG. 3). The effect of 25 and 50 nM PTK787 can be blocked by addition of rec VEGF to the cell cultures. In TF-1 cells inhibition of VEGFR signaling induces apoptosis (shown by PI DNA labeling) in a dose-dependent way up to 12% after overnight culture versus 5% in control wells. By treatment with rec VEGF₁₆₅ the fraction of apoptotic cells is again in the range of the control wells (4%).

Blocking VEGFR signaling with PTK787 decreases leukemic cell survival of HL-60 cells up to 1% and of TF-1 cells up to 10% (FIG. 4). As expected, the leukemic cell survival of K562 cells shows no decrease at all.

Although VEGFR-2 expression is not found with RT-PCR and VEGFR-1 expression could only be demonstrated in U937 and HL-60 cells, these data suggest that blocking VEGFR signaling induce apoptotic cell death which can be inhibited by addition of VEGF₁₆₅.

### Example 2

### Effect of VEGF and PTK787 on Patient Leukemic Cell Survival

In 6 primary AML samples the expression of VEGFR-1 and -2 as well as VEGF₁₆₅ is analyzed by RT-PCR (FIG. 1). In 5 of the 6 patients mRNA could be isolated and RT-PCR was performed. All 5 patients expressed various amounts of VEGF. Weak PCR bands for VEGFR-1 are found in 4 out of 5 patients. Patient 2, 3 and 6 express high amounts of VEGFR-2 transcripts, whereas patient 1 and 5 do not express VEGFR-2 at all (FIG. 1). Although VEGFR expression could not be assessed in all patient samples, all samples were tested for functional VEGFRs. Leukemic cell survival under serum free conditions demonstrated a large variation in the absolute number of viable cells after 24 hours in the different patient samples (median: 23.5×10³ viable cells; range: 6.6−78.1×10³ viable cells). With 5 ng/mL rec VEGF₁₆₅ leukemic cell survival is enhanced from 100% up to 143.3% (range: 114.2%-188.9%; p-value: 0.043), whereas addition of PTK787 results in increased cell death (median: 55.1% decrease; range: 28.2%-75.4% decrease; p-value: 0.043) (FIG. 5). Addition of exogenous VEGF₁₆₅ to the leukemic cell cultures with PTK787 (25 nM) could abrogate the effects of PTK787 to a median decrease in cell survival of 15% (range: 0%-35.5%) compared to 55.1% with PTK787 alone. So exogenous VEGF₁₆₅ rescues the effects of PTK787 with 73% of the total effect on cell death induced by PTK787.

### Example 3

### Effect of PTK787 and Conventional AML Drugs on Patient Leukemic Cell Survival

Leukemic cells of patients are incubated with different dosages of PTK787 and leukemic cellular drug resistance is assessed by a total cell kill assay. Results of in vitro resistance for PTK787 are summarized in Tablel and illustrated in FIG. 6. Dose-response curves (FIG. 6) as well as LC50 values for the individual patients (Table 1) are given.

Marked differences between individual patients are found. The expression of VEGFR-1 is weak in all patients, whereas the expression of VEGFR-2 is varying from no expression towards high expression levels. Patient samples with high VEGFR-2 expression and low VEGF expression (No. 2 and 3) are sensitive for PTK787 inhibition resulting in a high leukemic cell cytotoxicity, whereas patient 6 with high VEGFR-2 and VEGF expression demonstrates an intermediate result when PTK787 is added to the leukemic cell cultures. The most resistant sanmple (No. 1) shows high VEOF expression and no VEGFR-2 expression by RT-PCR. Although the numbers are small, these data suggest that the result of individual leukemic cytotoxicity upon PTK787 incubation is the result of VEGF together with VEGFR-2 expression levels.

The simultaneous use of PTK787 together with conventional chemotherapeutic drugs such as mitoxantrone and cytarabine results in an additive effect on leukemic cell cytotoxicity. In FIG. 7 as well as in Table 1 it is demonstrated that PTK787 leads to a dramatic lowering of dose-response curves for cytarabine and mitoxantrone, resulting in a decline of LC50 values for cytarabine and mitoxantrone with the addition of PTK787.

TABLE 1
LC50 values for various drugs in AML samples of different patients.
LC50 values given as numbers, in the presence of PTK787 and/or
combinations of cytarabine or mitoxantrone with PTK787.

| | Patient No. | LC50 value |
|---|---|---|
| PTK787 (nM) | 1 | >25 |
| | 2 | 7.3 |
| | 3 | 5.8 |
| | 4 | 25 |
| | 5 | 7.7 |
| | 6 | 14.5 |

| | | | PTK787 | PTK787 | PTK787 |
|---|---|---|---|---|---|
| Drug | Patient No. | LC50 value | 5 nM | 10 nM | 25 nM |
| Cytarabine | 1 | 400 | 260 | 250 | 35 |
| (μg/mL) | 2 | 5 | 0.6 | 0 | 0 |
| | 3 | 4.7 | 0.23 | 0 | 0 |
| | 4 | 1500 | 560 | 10 | 0.2 |
| | 5 | 3 | 0.24 | 0 | 0 |
| Mitoxantrone | 1 | 230 | 160 | 190 | 30 |
| (ng/mL) | 2 | 17.5 | 0.061 | 0 | 0 |
| | 3 | 43 | 0.4 | 0 | 0 |
| | 4 | 54 | 0.023 | 0 | 0 |
| | 5 | 18.6 | 0.061 | 0.0008 | 0 |

What is claimed:

1. A method of treating a warm-blooded animal having acute myeloid leukemia (AML), comprising administering to said animal a therapeutically effective amount of a compound of formula I 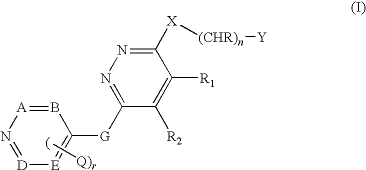
wherein
r is 0 to 2,
n is 0 to 2,
m is 0 to 4,
R₁ and R₂ (i) are lower alkyl or
(ii) together form a bridge in subformula I* 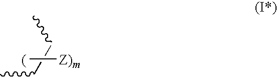
the binding being achieved via the two terminal carbon atoms, or
(iii) together form a bridge in subformula I** 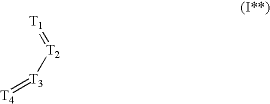
wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom, or the salt of such compound having at least one salt-forming group;
together or in combination with a conventional compound or compound mixture useful in AML treatment and optionally at least one pharmaceutically acceptable carrier.

2. A method of claim 1 wherein the compound of formula I is PTK787.

3. A method of claim 1 wherein the conventional compound useful in AML treatment is a topoisomerase II inhibitor, an antimetabolite, an antitumor antibiotic or a mixture of such compounds.

4. A method of claim 1 wherein the conventional compounds useful in AML treatment is selected from the group consisting of amsacrine, etoposide, teniposide, cytarabine, methotexate, mercaptopurine, mitoxantrone, dactinomycin, daunorubicin, doxorubicin, epirubicin, homoharringtonine, idarubicin, asparaginase, cyclophosphamide, gemtuzumab, other CD 33 monoclonal antibodies, ifosfamide, mesna, prednisone, topotecan, vincristine, and mixtures thereof.

5. A method according to claim 1 wherein the AML is resistant to conventional chemotherapy.

6. A method according to claim 1 wherein the warm-blooded animal is a human.

7. A method according to claim 6 wherein the human is a juvenile human.


SEQUENCE LISTING (10 sequences)

SEQ ID NO: 1 - DNA, 20 bp, Artificial
  sense primer for beta2-microglobulin
ccagcagagaatggaaagtc

SEQ ID NO: 2 - DNA, 20 bp, Artificial
  anti-sense primer for beta2-microglobulin
gatgctgcttacatgtctcg

SEQ ID NO: 3 - DNA, 27 bp, Artificial
  sense primer for VEGF
gagtgtgtgcccactgaggagtccaac

SEQ ID NO: 4 - DNA, 27 bp, Artificial
  anti-sense primer for VEGF
ctcctgcccggctcaccgcctcggctt

SEQ ID NO: 5 - DNA, 20 bp, Artificial
  sense primer for VEGFR-1
gagtcctttatcctggatgc

SEQ ID NO: 6 - DNA, 20 bp, Artificial
  anti-sense primer for VEGFR-1
acagagcccttctggttggt

SEQ ID NO: 7 - DNA, 18 bp, Artificial
  sense primer for VEGFR-2  first PCR
cgctgggagaaagaaccg

SEQ ID NO: 8 - DNA, 23 bp, Artificial
  anti-sense primer for VEGFR-2  first PCR
gctcactgccactctgattattg

SEQ ID NO: 9 - DNA, 26 bp, Artificial
  sense primer for VEGFR-2 nested PCR
tccgcgcctcctccttctctagacag

SEQ ID NO: 10 - DNA, 21 bp, Artificial
  anti-sense primer for VEGFR-2  nested PCR
ggccatcgctgcactcagtga